(19) United States
(12) Patent Application Publication
(10) Pub. No.: US 2013/0150699 A1
(43) Pub. Date: Jun. 13, 2013

(54) SCLERA SENSOR

(71) Applicants: Max Ostermeier, Seevetal (DE); Stefan Meyer, Hannover (DE)

(72) Inventors: Max Ostermeier, Seevetal (DE); Stefan Meyer, Hannover (DE)

(73) Assignee: IMPLANDATA OPHTHALMIC PRODUCTS GMBH, Hannover (DE)

(21) Appl. No.: 13/818,497

(22) PCT Filed: Aug. 15, 2011
(86) PCT No.: PCT/EP2011/064021

§ 371 Date: Feb. 22, 2013

(30) Foreign Application Priority Data
Aug. 25, 2010 (DE) 10 2010 035 294.2

(51) Int. Cl. A61B 3/16

(52) U.S. Cl. CPC A61B 3/16
USPC 600/398

(57) ABSTRACT

The invention relates to an implantable measuring device (1) for measuring the intraocular pressure at an ocular sclera (2), having pressure sensor means (5, 7) embedded in a pressure-transmitting housing (6) for biocompatible contacting of the ocular sclera (2), having at least one, preferably substantially flat, pressure sensor area (7) that allows for a reliable measurement of the intraocular pressure and is operatively easy to insert, in as much as possible, while avoiding the disadvantages of the prior art, wherein it is proposed that the housing (6) is configured with dimensionally stable elasticity.

### CROSS REFERENCE TO RELATED APPLICATIONS

This application is the National Phase Application under 35 USC §371 of International Application No. PCT/EP2011/064021, filed Aug. 15, 2011, which claims priority to German Patent Application 10 2010 035 294.2, filed Aug. 25, 2010.

### BACKGROUND

A. Technical Field

The present invention relates to an implantable measuring device for measuring the intraocular pressure at an ocular sclera, having pressure sensor means embedded in a pressure-transmitting housing for biocompatible contacting of the ocular sclera, having at least one, preferably substantially flat, pressure sensor area.

B. Background of the Invention

A measuring device of this type is known in the art from DE 10 2004 056 757 A1. The measuring device allows for measuring the intraocular pressure from an easily accessible location on the eye, in fact extrasclerally, meaning via placement of the device against the ocular sclera. In contrast to intraocular measuring devices for measuring the intraocular pressure, the measuring device is placed on the ocular sclera below the conjunctiva. Contrary to intraocular measuring methods, the use of measuring devices for the extraocular measurement of intraocular pressure according to this class has the advantage that the implantation of the measuring device is less invasive and can therefore also be used in the context of a routine procedure, solely for the implantation of the measuring device. In contrast, intraocular implants are customarily only inserted when, as is typical in surgical cataract treatment, eye surgery is necessary anyway.

Known implantable measuring devices for the extraocular measurement of intraocular pressure according to DE 10 2004 056 757 A1 are reliant, in terms of the measuring principle that is applied for the pressure measurement, on a microchip, which contains a capacitive pressure sensor in addition to suitable electronics and is encased in a flexible envelope filled with a gel or an oil on silicone basis. According to the cited prior art, the known measuring device uses a plate, for example, configured as an acrylic plate, that is fixed in place on the external area of the ocular sclera in the region of the pars plana between two eye muscles, and wherein the plate serves as an abutment during the pressure measurement. Disadvantageously, the known measuring device provides that during the fixation of the same on the ocular sclera, while using the acrylic plate as an abutment, the fluid-filled envelope, containing the pressure sensor in oil or gel and lacking in dimensional stability, is pressed against the ocular sclera with the aid of the acrylic plate applying a press-on force that depends on the concrete fixation and is therefore variable. A pressure measurement is thus, disadvantageously, only possible after carrying out complex two-point or multiple-point calibrations. In fact, the pressure inside the oil- or gel-filled envelope is composed of the force that is to be measured as a dimension of the intraocular pressure acting upon the content of the flexible envelope via the ocular sclera and the force that is applied when the envelope is pressed against the ocular sclera.

Furthermore, due to the configuration of the known measuring device in form of, what is in principle, a flexible cushion containing a fluid, the contact area between this cushion and the ocular sclera, which is effective for the transmission of pressure into the cushion and thereby onto the pressure sensors contained therein, is also variable, depending on the type of the concrete fixation and the thus resulting press-on force. Correspondingly, if at all, any defined pressure measurement can only be achieved at a disadvantage following a corresponding complex calibration of the measured values. In addition, the apparatus that must be implanted for the measurement of the intraocular pressure on the ocular sclera is disadvantageously complex by comparison.

### SUMMARY OF THE INVENTION

With this background as outlined above, it is the object of the present invention to specify an implantable measuring device for measuring the intraocular pressure, a device of the kind as described in the introduction that will allow for a reliable measurement of the intraocular pressure while avoiding the disadvantages of the prior art and with optimal operative ease of insertability.

This object is achieved according to the invention with an implantable measuring device of the kind as outlined in the introduction having a housing that is configured with dimensionally stable elasticity. Advantageously, according to the invention, the housing can be provided with a defined contour for making contact with the ocular sclera, which is substantially maintained in the presence of the usual pressure and force conditions that occur on the ocular sclera in connection with the intraocular pressure measurement. The selection of an elastic housing therein is distinct from the flexible housing in form of a fluid- or gel-filled cushion as known in the art. Advantageously, it is possible to substantially simplify the calibration of the measurement if it is possible to achieve defined reference areas for transmitting the intraocular pressure from the ocular sclera to the pressure-transmitting housing based on a preset external contour of the housing. An elastic housing therein has the advantage that, while there occurs a transmission of the intraocular pressure from the ocular sclera to the pressure-transmitting housing and thereby the pressure sensor that is embedded therein, the external contour of the housing, however, is essentially maintained intact. Naturally, for carrying out the measurement, it is crucial that the measuring device according to the invention is fixed in place in a suitable manner on the ocular sclera.

In an advantageous configuration of the invention, a contact area of the housing that is intended to make contact with the ocular sclera has a concave surface contour that is substantially adjusted accordingly to provide a form closure with the ocular sclera. Said shaping of the contact area of the housing with the ocular sclera makes it possible for the measuring device according to the invention to be fixed on the ocular sclera, wherein, as a matter of principle, no press-on force is necessary for the fixation. Correspondingly, according to the invention, the fastening of the implantable measuring device according to the invention can thus occur, in principle, without any of the press-on forces that falsify the pressure measurement.

A special advantageous embodiment of the invention provides that the surface contour of the contact area is configured such as to achieve a form closure on the average ocular sclera of a given patient group for whom the measuring device is to be used. According to this embodiment, it is possible to take advantage of the benefit that the variability of the surface contours of the ocular sclera is minimal within certain patient groups. Therefore, the invention provides that envisioning one configuration for adult patients and one configuration for adolescent patients can already suffice. In each case, according to the invention, it is possible to advantageously manufacture a measuring device according to the invention in series for a collective of patients, without any requirement for individualized adjustment.

To preclude measuring artifacts, it is possible by way of an advantageous configuration of the invention, to shape a curvature of the contact surface as smaller than a curvature of the ocular sclera in the contact area. Assuming a ball shape, the area radius of the contact surface is greater than the radius of the ocular sclera in the contact area. According to this configuration of the invention, the measuring device according to the invention is pressed against the ocular sclera with a small press-on force. The invention envisions, however, for the press-on force to be captured by the structures surrounding the measuring device because the ocular sclera adjusts itself to the contour of the implant. The press-on force that is applied, due to the fixation of the measuring device on the ocular sclera by means of surgical sutures, is also reduced by the fact that the area radius of the concave contour of the implant is configured as somewhat larger than the radius of the area that is to be contacted on the eye.

Provided a further advantageous embodiment of the measuring device according to the invention envisions for the contact area to include a measuring area portion in a vicinity of the pressure sensor means having an essentially flat surface contour, a precisely defined pressure-sensitive area is achieved for the pressure measurement as a prerequisite for the implementation of a true pressure measurement, contrary to a simple force measurement. In fact, during the placement of the measuring device according to the invention corresponding to this special configuration on the ocular sclera, the ocular sclera is, as provided by the invention, relaxed only in the portion of the measuring area by applying a defined force, such that the intraocular pressure is transmitted via the measuring area portion to the housing, and thereby to the pressure sensor means embedded therein. Due to the fact that, according to the present implementation of the invention, the contact area of the housing has, in particular, a surface contour that is basically adjusted to the ocular sclera, which is only flat in the area of the portion of the measuring area, the measuring area portion can always be placed in a defined manner when inserting the implantable measuring device according to the invention, such that a relaxed defined zone of the ocular sclera is achieved. In fact, the flat measuring portion is pressed upon the ocular sclera until the portions of the contact area of the housing that are adjacent to the flat measuring portion hug the ocular sclera in the way of a form closure. According to the invention, this geometry allows for generating a defined relaxed zone inside which the ocular sclera is relaxed. On the other hand, the portion of the area that transmits pressure is always limited to the area of the measuring portion, such that the evaluation of defined, reproducible conditions is ensured for any measurement. The press-on force by which the measuring area portion is pressed against the ocular sclera naturally depends, corresponding to the measurement principle, on the intraocular pressure within the ocular sclera.

A further preferred embodiment of the invention provides that a face normal of the measuring area portion is arranged essentially parallel relative to a face normal of the pressure-sensing area. For example, a microchip comprising the pressure-sensor means is expediently embedded such in the elastic housing according to the invention that the pressure-sensing area is aligned parallel relative to the area of the measuring area portion in the region of the contact area of the housing. A transmission of the intraocular pressure from the ocular sclera via the measuring area portion into the housing thus results in this way advantageously in linear deflections of the pressure sensor means, particularly if the same is configured as a pressure membrane. The pressure sensors in the context of the present invention can be configured as capacitive or piezoresistive, wherein the use of a pressure membrane is determinative for the linearity of the measurement.

According to the invention, an expansion of the measuring area portion can correspond substantially to an expansion of the pressure-sensing area. Artifacts and boundary effects during the pressure measurement can thus be advantageously and effectively minimized.

Especially advantageously, the contact area in the context of the configuration according to the invention includes a neutralization area portion that is adjacent to the measuring area portion surrounding the same in a ring-type fashion and having a substantially flat surface contour. The term “surrounded in a ring-type fashion” stands therein for any kind of closed areal form. In particular, this term is not intended to limit neutralization area portions to a circular shape. The advantageous effect of the neutralization area portion lies in the minimization, or elimination altogether, of artifacts and boundary effects in the transitional area from the flattened area to the region that is adjusted to the ocular sclera.

Especially advantageously according to the invention, the housing is configured as rubber-elastic. Silicone rubber has proved especially suitable for the manufacture of the housing within the context of the invention. When using a material for the housing, considering the biocompatibility of the same is crucial, same as the suitability thereof for transmitting pressure to the pressure sensor that is embedded in the housing, wherein, according to the invention, any elasticity must additionally be envisioned in the sense of providing form stability as well.

A preferred embodied example of the invention provides for fastening means in order to attach the measuring device on the ocular sclera. Advantageously, it is thus possible to achieve, for example by suturing, a fixation on the ocular sclera without any need for a separate component that serves simultaneously as an abutment such as in the form of an acrylic plate, as required by the referenced prior art.

In a special embodiment of the invention, the fastening means can comprise barbed hooks.

It has proved particularly advantageous for the fastening means to comprise eyelet-type openings for running a suture or the like through the same that is connected to the ocular sclera. It is especially advantageous in this context to envision pairs of eyelet-type openings, particularly two or four eyelet-type openings, which are disposed in pairs and symmetrically relative to each other. This configuration provides that the device according to the invention can be fixed on the ocular sclera in such a way that any fixation forces are equalized such that a connection of the kind of a form closure is achieved between the contact surface and the ocular sclera.

In particular, it is possible to provide eyelets of a seam for surgical sutures on the measuring device according to the invention.

A variant of the invention envisions that the contact area (8) be provided with a gluing means for creating a glued connection with the ocular sclera, and/or the same is made of a material with self-adhesive properties for placement upon the ocular sclera.

Should, according to an advantageous variant of the invention, the fastening means be embodied as a fastening module that is separate from the housing and connected thereto, particularly by mechanical means, wherein the fastening module is configured, in particular, in the way of a hollow cylinder and/or preferably manufactured of a material of a lesser elasticity than the housing, a simplified manufacture is possible. In fact, the fastening module can be manufactured of a different, for example, more solid material than the housing. According to this variant of the invention, the housing must, taken by itself, not include fastening means as well in order to be fastened to the ocular sclera and can therefore be optimized in terms of the configuration thereof for the needs of the actual, artifact-free intraocular pressure measurement in the sense as outlined above.

An improvement of the invention provides for embedding a telemetry means in the pressure-transmitting envelope, particularly comprising an inductive coil. In particular, it is possible to use an electric telemetry means integrated in the same chip that also contains the pressure sensor. The electronic components of the telemetry means can preferably be in direct electronic contact with the inductive coil by means of bonding. The at least one pressure sensor therein can be located on the one side, and the inductive coil on the other side of the flat body of the microchip. In the alternative, within the scope of the invention, it is possible to envision the pressure sensor and the inductive coil also on the same surface side of the body of the microchip. The coil can be configured as a flat coil or as a deformable, wound-up coil. The coil can be manufactured by means of technical methods that are specific for use in micro-systems, such as, for example, by microgalvanic means, particularly photolithography. The query of the measured pressure values as detected by the measuring device according to the invention can be implemented by a telemetric query means that communicates with the telemetry means of the measuring device. Typically, it makes sense for the telemetric query means to be disposed outside of the ocular sclera and/or externally of the eye, particularly at an extracorporeal location.

### BRIEF DESCRIPTION OF THE DRAWINGS

The invention shall be described in an exemplary manner below in reference to a preferred embodied example and in reference to a drawing, wherein further advantageous details can be derived from the drawing.

Parts that are identical in terms of the function thereof are identified by identical reference signs.

The figures of the drawing show in detail as follows:

FIG. 1—a schematic side view of an implantable measuring device according to a first embodied example of the invention that is laterally fixed on the ocular sclera;

FIG. 2—an enlarged cut-out of a section marked II according to 1;

FIG. 3—a perspective view from a transverse-top angle onto the side of the second embodied example of the invention that is pointed away from the ocular sclera;

FIG. 4—a cut-out of a top view in direction of an arrow IV onto the side of the ocular sclera that is directed toward the measuring device according to FIG. 3.

### DETAILED DESCRIPTION OF THE PREFERRED EMBODIMENTS

FIG. 1 is a schematic representation of an implantable measuring device 1 that is fixed laterally on an ocular sclera 2 in the vicinity of the pars plana or behind the pars plana. The figure further shows by way of a schematic representation the cornea 3 of the ball of the eye 4. The intraocular pressure that is prevalent inside the ocular sclera 2 is schematically symbolized by arrows.

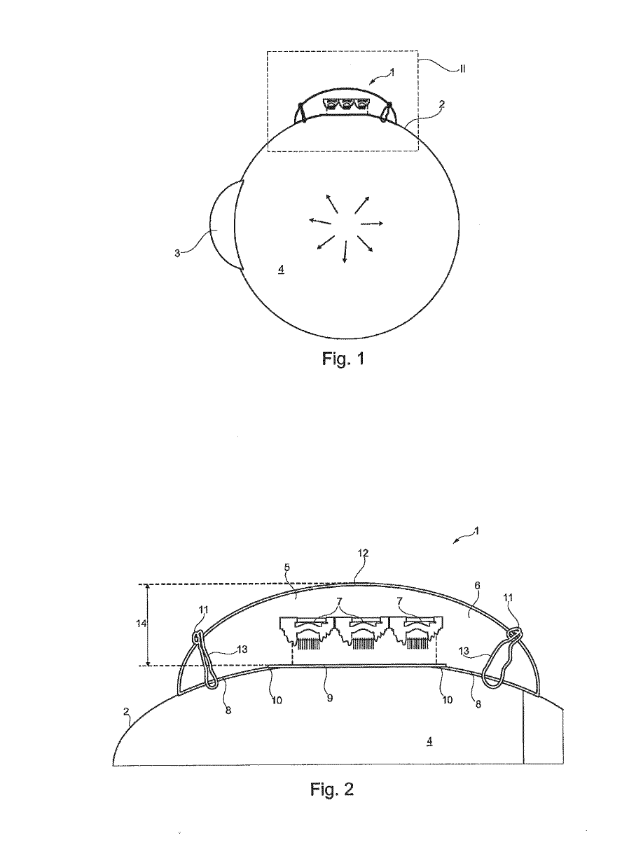

FIG. 2 shows an enlargement of a cut-out of the region specified as II in FIG. 1 for a more detailed explanation of the setup of the implanted measuring device 1. As can be seen in FIG. 2, the implantable measuring device is made up essentially of an ASIC 5 that is embedded in a housing 6. The housing 6 is made, for example, of silicone rubber or another biocompatible rubber-elastic material.

The ASIC 5 is known in the art. The setup of the same is described, for example, in DE 10 2004 056 757 A1. In particular, the ASIC 5 as depicted in FIG. 2 includes pressure membrane 7 that converts, as known from the prior art, deflections into a measured value, for example following a capacitive measuring process. Furthermore, the ASIC 5 contains, as known from the prior art, a telemetry means and an inductive coil for the wireless transmission of measured pressure values to an extracorporeal telemetric query means. The scope of the invention provides that the pressure sensor can be disposed equally as separate of the telemetry means and/or of other electronic components on a separate substrate. Therefore, the ASIC must not necessarily contain a pressure sensor. Instead, within the scope of the invention it is also possible to configure a pressure sensor separately of an ASIC and connect the same thereto by means of a cable connection or in another separate manner that is known in the art.

A contact surface 8 of housing 6 is placed on the ocular sclera 2 of the eyeball 4. The housing 6 therein is, in essence, dimensionally stable and elastic at the same time. In terms of the basic form thereof, the contact surface 8 is concave and adjusted to the ocular sclera 2, such that in the placed state, as demonstrated in the figures, the contact surface 8 is connected with the ocular sclera 2 by way of a form closure. Contact surface 8, however, is configured substantially as flat according to the invention in the area of the measuring area portion 9. The alignment of the flat measuring area portion 9 is selected as being parallel relative to the alignment of the pressure membrane 7.

In the side view of the figures left and right, adjacent to the measuring area portion 9, the contact surface 8 of the housing, made of silicone rubber 6, includes a flat neutralization area portion 10. The neutralization area portion 10 surrounds the measuring area portion 9 in the manner of a ring 9 and constitutes the transition between the measuring area portion 9 and the contact surface 8 that is adjusted to the contour of the ocular sclera 2. The measuring area portion 9 corresponds regarding the extension thereof to the extension of the area of the ASIC 5 comprising the pressure membrane 7.

The measuring device 1 includes seam eyelets 11 in the rubber-elastic housing 11 that are fixed in place on the external surface 12 of the housing 6, opposite relative to the contact surface 8.

As indicated schematically only in the figures, the measuring device 1 is fixed via the seam eyelets 11 on the external surface 12 of the housing 6 and surgical sutures 13 to the ocular sclera 2. The seam eyelets 11 for receiving the surgical sutures 13 are disposed symmetrically in such a way that an even fixation of the housing 6 on the ocular sclera is ensured, wherein there exists substantially a form closure connection with the ocular sclera 2 in the area of the contact surface 8.

A deflection of the ocular sclera 2 occurs only in the flattened region of the measuring area portion 9 as well as of the neutralization area portion 10, which surrounds the measuring area portion 9 in a ring-type fashion. Said deflection of the ocular sclera 2 results in an elimination of the pressure-inducing tension on the ocular sclera 2, such that the ocular sclera 2 is not in a state of tension. Correspondingly, the pressure membrane 7 can, taking into consideration the fixedly defined measuring area portion 9, measure the intraocular pressure prevalent in the ball of the eye 4 that is transmitted via the relaxed ocular sclera 2 at a right angle relative to the tangent of the ball of the eye 4.

Since, according to the invention, the measuring device is used with a separate abutment, such as, for example, specified in DE 10 2004 056 757 A1, no extreme restrictions are applicable regarding a height 14. It is therefore advantageously possible for the setup of the ASIC 5 to be of a greater height as well than, for example, 0.7 mm, which differs from the setup of the known ASICs according to DE 10 2004 056 757 A1.

The measuring area portion 9 can, for example, advantageously have a diameter of only approximately 2 mm. Due to the fact that a flattening of the ocular sclera 2 occurs only in the area of the measuring area portion 9, it is possible to keep the tension forces that are in effect upon the surgical sutures 13 advantageously minimal in order to avoid a detachment of the measuring device 1 according to the invention from the ocular sclera 2, or to render such a detachment less likely.

The total diameter of the flattened region constituted by the combined measuring area portion 9 and the neutralization area portion 10 is, on the other hand, preferably approximately 3 mm according to this embodiment of the invention.

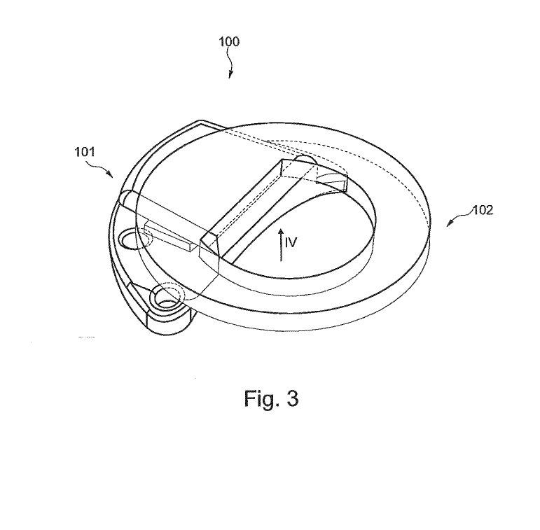

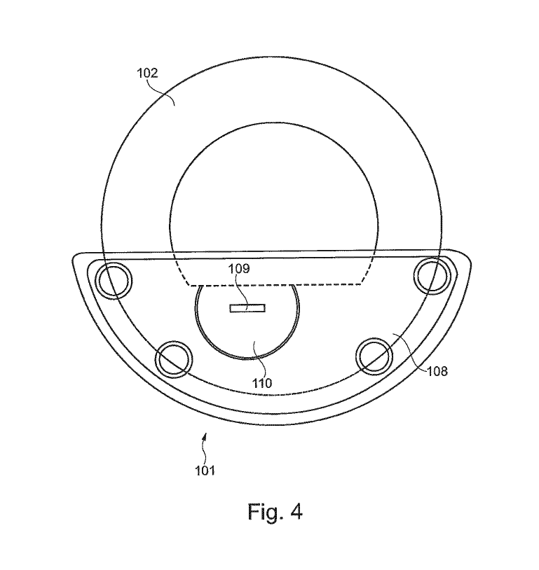

FIG. 3 shows an alternate configuration of an implantable measuring device according to the invention 100 by way of a perspective view in the direction of the side that is pointed away from the ocular sclera. As discernable in FIG. 3, the measuring device 100 for measuring the intraocular pressure is configured in two parts contrasting the same from the embodied example as represented in FIGS. 1 and 2. Specifically, on the one hand, the measuring device 100 comprises a measuring module 101 and, on the other hand, a fastening module 102 that is configured separately of the measuring module 101. Regarding the implemented functions and essential properties, the measuring module 101 is configured correspondingly relative to the measuring device 1 as outlined with regard to FIGS. 1 and 2 above.

In particular FIG. 4, which shows a flat top view in the direction of arrow IV according to FIG. 3 onto the side of the measuring module 101 that is directed toward the ocular sclera, it is discernable that the bottom side includes a contact area 108. The contact surface 108 of the measuring module 101 of the measuring device 100 in the embodied example as presently described is, analogous relative to the contact surface 8 with regard to the embodied example of the invention as described in FIGS. 1 and 2, adjusted, in terms of the basic shape thereof, in a concave manner to the shape of the ocular sclera, such that, in the placed state of the contact surface 108 on the ocular sclera, the contact surface 108 is connected with the ocular sclera by way of a form closure.

Analog to the contact surface 8 in the above-described first embodiment, the contact surface 108 according to the invention includes, however, a measuring area portion 109 that is substantially configured as flat. The alignment of the flat measuring area portion 109 is parallel relative to the alignment of the pressure membrane, which is not visible in the figure. The measuring area portion 109 is also surrounded by a neutralization area portion 110 of a flat configuration, as can be seen in FIG. 4.

The measuring module 101 is manufactured of silicone rubber, for example, or of another biocompatible, rubber-elastic material. The determinative aspect for the material selection is that the measuring module 101 is dimensionally stable while elastic in order to ensure the shaping of the contact surface and of the measuring area portion 109, as well as of neutralization area portion 110. The shape of the contact surface 108 of the measuring module 101 that is adjusted to the curvature of ocular sclera can be seen especially nicely in FIG. 3. The fastening module 102 has the shape of a hollow cylinder. As pointed to in FIGS. 3 and 4, the fastening module 102 is mounted on the side of the measuring module 101 that is directed away from the ocular sclera and rests thereupon. The fastening module 102 of the shape of a hollow cylinder protrudes from the measuring module 101 therein, which has in the vertical projection in the presently described embodiment the shape of a circular segment. In a preferred configuration, the fastening module 102 is manufactured of a harder material than the measuring module 101. In particular, according to the invention, the fastening module 102 can be manufactured of acrylic.

In the placement area of the fastening module 102 on the measuring module 101, there exists a mechanical connection, which is not shown in further detail in the figures. Similarly, the connection between the fastening module 102 and the measuring module 101 can also be embodied in another manner, for example, by gluing or the like, without leaving the scope of the invention.

When implanting the measuring device according to the invention 100 in correspondence with the configuration as represented in FIGS. 3 and 4, a fastening on the ocular sclera can be achieved in that the portion of the fastening module 102 that protrudes the measuring module 101 is fastened on the ocular sclera in a suitable manner that is known as a matter of fact to a person skilled in the art. Due to the fastening of the fastening module 102 on the ocular sclera there occurs a defined press-on action of the measuring module 101 on the ocular sclera, analogously relative to the manner that was sketched with regard to the embodied example as represented in FIGS. 1 and 2. The special area shaping of the contact surface 108 with the measuring area portion 109 and the neutralization area portion 110 surrounding the measuring area portion 9 in turn ensure that a defined flat area is generated having the advantages according to the invention as described above.

The two-part configuration of the measuring device 100 according to FIGS. 3 and 4 is able to advantageously achieve a functional decoupling of the fastened connection of the measuring module 101 on the ocular sclera from the technical requirements with regard to the measurement of intraocular pressure. Correspondingly, as mentioned previously, the fastening module 102 can be made of a harder material than the measuring module 101. Nevertheless, based on the shaping of the contact surface 108 that touches the ocular sclera of the measuring module 101, it is possible to advantageously achieve a pressure measurement that is free of artifacts, in as much as that is possible.

Correspondingly, two embodied examples of an implantable measuring device according to the invention have thus been proposed; they are characterized by easy construction and allow for the extrascleral measurement of intraocular pressure, thus excluding system-related measurement errors as are possible with the prior art.

### LIST OF REFERENCE SIGNS

1 Measuring device
2 Ocular sclera
3 Cornea
4 Ball of the eye
5 ASIC
6 Housing
7 Pressure membrane
8 Contact surface
9 Measuring area portion
10 Neutralization area portion
11 Seam eyelet
12 External surface
13 Surgical suture
14 Height
100 Measuring device
101 Measuring module
102 Fastening module
108 Contact surface
109 Measuring area portion
110 Neutralization area portion

What is claimed is:

1. An implantable measuring device for measuring the intraocular pressure on an ocular sclera, having comprising:
a pressure-transmitting housing configured with dimensionally stable elasticity; and
a pressure sensor means embedded in the pressure-transmitting housing for biocompatible contacting of the ocular sclera, having at least one, substantially flat pressure sensor area.

2. The measuring device of claim 1, wherein a contact area of the housing is provided for making contact with the ocular sclera has a concave surface contour that is adjusted to achieve a form closure with the ocular sclera.

3. The measuring device of claim 2, wherein the surface contour of the contact area is shaped for achieving a form closure with an average ocular sclera of a patient group for which the measuring device is to be used.

4. The measuring device of claim 1, wherein a curvature of the contact area is smaller than a curvature of the ocular sclera in the contact area.

5. The measuring device of claim 1, wherein the contact area includes a measuring area portion with a substantially flat surface contour within an area surrounding the pressure sensor.

6. The measuring device of claim 1, wherein a face normal to the measuring area portion is arranged substantially parallel relative to a face normal to the pressure sensor area.

7. The measuring device of claim 1, characterized in that wherein the expansion of the measuring area portion corresponds substantially to an expansion of the pressure sensor area.

8. The measuring device of claim 1, wherein the contact area includes a neutralization area portion that is bordering the measuring area portion, surrounding the same in a ring-type manner and having a substantially flat surface contour.

9. The measuring device of claim 1, wherein the housing is configured as rubber-elastic.

10. The measuring device of claim 1, a fastening mechanism is provided for fastening to the ocular sclera.

11. The measuring device of claim 1, wherein the fastening mechanism comprises barbed hooks.

12. The measuring device of claim 1, wherein the fastening mechanism comprises an eyelet-type openings for passing a suture through the same that is connected to the ocular sclera.

13. The measuring device of claim 12, wherein the eyelet-type openings are arranged symmetrically relative to each other and in pairs, particularly two or four in number.

14. The measuring device of claim 10, wherein the fastening mechanism is developed as a fastening module that is separate from the housing and connected thereto, particularly by mechanical means, wherein, the fastening module is configured in the way of a hollow cylinder and/or manufactured of a material of a lesser elasticity than the housing.

15. The measuring device of claim 1, wherein the contact area is provided with an adhesive agent for achieving a glued connection with the ocular sclera and/or manufactured of a material that is self-adhesive on the ocular sclera.

16. The measuring device of claim 1, wherein a telemetry module is embedded in the pressure-transmitting housing, particularly comprising an inductive coil.
